(19) United States
(12) Patent Application Publication
(10) Pub. No.: US 2008/0281099 A1
(43) Pub. Date: Nov. 13, 2008

(54) PROCESS FOR PURIFYING VALACYCLOVIR HYDROCHLORIDE AND INTERMEDIATES THEREOF

(76) Inventors: Mayur Devjibhai Khunt, Junagadh (IN); Bojja Ramachandra Reddy, Hyderabad (IN); Keshava Naveen Kumar Reddy, Hyderabad (IN); Vedantham Ravindra, Hyderabad (IN); Sripathi Somaiah, Nalgonda (IN); Sundaraneedi Veera Madhavi, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 12/116,277

(22) Filed: May 7, 2008

Related U.S. Application Data
(60) Provisional application No. 61/029,460, filed on Feb. 18, 2008.

(30) Foreign Application Priority Data
May 7, 2007 (IN) 961/CHE/2007

(51) Int. Cl. C07D 473/18

(52) U.S. Cl. 544/276

(57) ABSTRACT

The invention relates to processes for the purification of valacyclovir hydrochloride and intermediates thereof.

### INTRODUCTION TO THE INVENTION

The present invention relates to processes for the purification of valacyclovir hydrochloride and intermediates thereof.

Valacyclovir hydrochloride, the hydrochloride salt of the L-valyl ester of the antiviral drug acyclovir, has the chemical names (2-[2-amino-1,6-dihydro-6-oxo-9H(purin-9-yl)methoxy]ethyl-L-valinate hydrochloride, or L-valine, 2-[(2-amino-1,6-dihydro-6-oxo-9H-purin-9-yl)methoxy]ethyl ester, monohydrochloride, and may be depicted by structural Formula I.

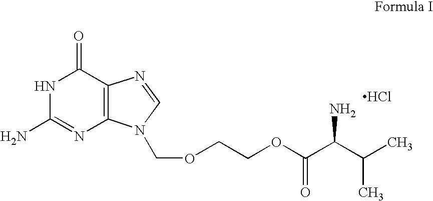

An aspect of the present invention relates to a purification method for 2-[(2-amino-6-oxo-1,6-dihydro-9H-purin-9yl)methoxy]ethyl-N-[(benzylox y)carbonyl]-L-valinate having the structure shown in Formula II, which is an intermediate in the synthesis of valacyclovir hydrochloride.

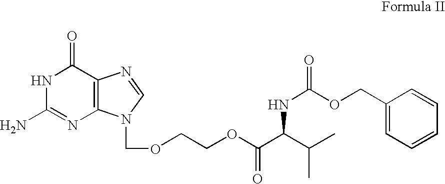

Valacyclovir hydrochloride is useful for the treatment of herpes simplex and varicella-zoster viral infections in humans, and is commercially available in pharmaceutical products sold by GlaxoSmithKline using the trademark VALTREX, as caplets containing the equivalent of 500 mg or 1 gram of valacyclovir.

U.S. Pat. No. 4,957,924 discloses valacyclovir hydrochloride, its pharmaceutical composition and a method of treatment using this composition.

International Application Publication No. WO 2006/0029253 A1 discloses the isolation and process for the preparation of an N-formyl valacyclovir impurity and use of the N-formyl valacyclovir impurity as a reference standard in the quantitative analysis of valacyclovir or its pharmaceutically acceptable salts.

It is well known in the art that, for human administration, safety considerations require the establishment, by national and international regulatory authorities, of very low limits for identified, but toxicologically uncharacterized impurities, before an active pharmaceutical ingredient (API) product is commercialized. Typically, these limits are less than about 0.15 percent by weight of each impurity. Limits for unidentified and/or uncharacterized impurities are obviously lower. Therefore, in the manufacture of an API, high purity of the products is required before commercialization, as is the purity of the active agent in the manufacture of formulated pharmaceuticals.

It is also known in the art that impurities in an API may arise from degradation of the API itself, which is related to the stability of the pure API during storage, and the manufacturing process, including the chemical synthesis. Process impurities include unreacted starting materials, chemical derivatives of impurities contained in starting materials, synthetic byproducts, and degradation products.

In addition to stability, which is a factor in the shelf life of the API, the purity of the API produced in the commercial manufacturing process is clearly a necessary condition for commercialization. Impurities introduced during commercial manufacturing processes must be limited to very small amounts, and are preferably substantially absent. For example, the ICH Q7A guidance for API manufacturers requires that process impurities be maintained below set limits by specifying the quality of raw materials, controlling process parameters, such as temperature, pressure, time, and stoichiometric ratios, and including purification steps, such as crystallization, distillation, and liquid-liquid extraction, in the manufacturing process. In the United States, the Food and Drug Administration guidelines recommend that the amounts of some impurities be limited to less than 0.1 percent.

As is known by those skilled in the art, the management of process impurities is greatly enhanced by understanding their chemical structures and synthetic pathways, and by identifying the parameters that influence the amount of impurities in the final product. Therefore, impurities in an API result primarily from one of two sources: (1) the manufacturing process or synthesis of the API; and (2) from the degradation of the API itself.

Once pure valacyclovir hydrochloride is obtained, i.e., the valacyclovir hydrochloride is substantially free of process impurities, or the process impurities are present in very small, limited amounts at the end of its manufacturing process. Degradation impurities, which are related to stability during storage, are the primary source of impurities, as long as contamination is prevented. Manufacturers are required by national and international laws and regulations to submit appropriate documentation to regulatory authorities, proving stability of both the API and formulated pharmaceutical products. It is therefore known in the art that stability of the API itself is a necessary condition for commercialization. See, e.g., the ICH Q7A guidance for API manufacturers.

Generally, side products, by-products, and adjunct reagents (collectively “impurities”) are identified spectroscopically and/or with another physical method, and then associated with a peak position, such as that in a chromatogram, or a spot on a TLC plate. Thereafter, the impurity may be identified, e.g., by its relative position in the chromatogram, where the position in a chromatogram is conventionally measured in minutes between injection of the sample on the column and elution of the particular component through the detector. The relative position in the chromatogram is known as the “retention time.”

As is known by those skilled in the art, the management of process impurities is greatly enhanced by understanding their chemical structures and synthetic pathways, and by identifying the parameters that influence the amount of impurities in the final product.

There remains a continuing need for simple, industrially feasible, inexpensive, scaleable and safe-to-handle processes for the synthesis of valacyclovir hydrochloride with high purity.

### SUMMARY OF THE INVENTION

The present invention relates to processes for the purification of valacyclovir hydrochloride and intermediates thereof.

In one aspect, there is provided the compound 2-[(2-amino-6-oxo-1,6-dihydro-9H-purin-9yl)methoxy]ethyl-N-[(benzyloxy)carbonyl]-L-valinate of Formula II, which contains a low concentration of its D-isomeric impurity.

In another aspect, there are provided processes for the purification of 2-[(2-amino-6-oxo-1,6-dihydro-9H-purin-9yl)methoxy]ethyl-N-[(benzylox y)carbonyl]-L-valinate of Formula II, providing a product that contains a low concentration of the D-isomeric impurity, an embodiment of a process including:

1 ) providing a solution of 2-[(2-amino-6-oxo-1,6-dihydro-9H-purin-9-yl)methoxy]ethyl-N-[(benzyloxy)carbonyl]-L-valinate of Formula II in a suitable organic solvent or mixture of solvents under suitable conditions;

2) cooling the reaction solution of step 1) to suitable temperatures for crystallization of the solid, and/or adding a suitable antisolvent or mixture of antisolvents to the solution of step 1) to form crystals under suitable conditions; and

3) recovering a solid formed in step (2) to afford the desired pure compound 2-[(2-amino-6-oxo-1,6-dihydro-9H-purin-9yl)methoxy]ethyl-N-[(benzyloxy) carbonyl]-L-valinate of Formula II.

In a further aspect, there is provided the compound [(2-amino-6-oxo-1,6-dihydro-9H-purin-9yl)methoxy]ethyl-N-[(benzyloxy)carbonyl]-L-valinate of Formula II obtained by the above process with a chiral purity greater than or equal to about 97% w/w as determined by high performance liquid chromatography (“HPLC”).

In a still further aspect, there are provided processes for the purification of valacyclovir hydrochloride of Formula I, an embodiment of a process including:

1 ) providing a solution of valacyclovir hydrochloride in a suitable solvent or mixture of solvents under suitable conditions;

2) cooling the reaction solution of step 1) to suitable temperatures for crystallization of the solid, and/or adding a suitable antisolvent or mixture of antisolvents to the solution of step 1) to form crystals under suitable conditions; and

3) recovering a solid formed in step 2) to afford the desired pure compound.

In yet another aspect, there is provided pure valacyclovir hydrochloride of Formula I having chiral purity greater than or equal to about 97% w/w as determined by high performance liquid chromatography (HPLC).

In another aspect, there are provided pharmaceutical compositions comprising valacyclovir or its pharmaceutically acceptable salt and at least one pharmaceutically acceptable carrier.

The processes of the present invention are simple, cost effective, eco-friendly, and reproducible, afford high yields and purity, and are well suited for commercial production.

### DETAILED DESCRIPTION OF THE INVENTION

In an embodiment, there is provided the compound 2-[(2-amino-6-oxo-1,6-dihydro-9H-purin-9yl)methoxy]ethyl-N-[(benzyloxy)carbonyl]-L-valinate of Formula II, which is substantially free of its D-isomeric impurity.

In another embodiment, there are provided processes for the purification of 2-[(2-amino-6-oxo-1,6-dihydro-9H-purin-9yl)methoxy]ethyl-N-[(benzylox y)carbonyl]-L-valinate of Formula II, an embodiment of a process including:

1 ) providing a solution of 2-[(2-amino-6-oxo-1,6-dihydro-9H-purin-9-yl)methoxy]ethyl-N-[(benzyloxy)carbonyl]-L-valinate of Formula II in a suitable organic solvent or mixture of solvents under suitable conditions;

2) cooling the reaction solution of step 1) to suitable temperatures for crystallization of the solid, and/or adding a suitable anti-solvent or mixture of anti-solvents to the solution of step 1) to form crystals under suitable conditions; and

3) recovering a solid formed in step 2) to afford the desired pure compound 2-[(2-amino-6-oxo-1,6-dihydro-9H-purin-9yl)methoxy]ethyl-N-[(benzyloxy) carbonyl]-L-valinate of Formula II.

Step 1) involves providing a solution of 2-[(2-amino-6-oxo-1,6-dihydor-9H-purin-9yl)methoxy]ethyl-N-[(benzyloxy) carbonyl]-L-valinate of Formula II in a suitable solvent or mixture of solvents under suitable conditions.

Suitable organic solvents that may be used include but are not limited to: alcoholic solvents such as methanol, ethanol, isopropyl alcohol, n-butanol, tertiary-butyl alcohol, and the like; ketonic solvents such as acetone, ethyl methyl ketone, methyl isobutyl ketone and the like; esters such as ethyl acetate, n-propyl acetate, isopropyl acetate, n-butyl acetate, tertiary-butyl acetate and the like; nitrile solvents such as acetonitrile, propionitrile and the like; halogenated solvents such as dichloromethane, ethylene dichloride, chloroform and the like; aprotic polar solvents such as N,N-dimethylformamide (DMF), dimethylsulfoxide (DMSO), N,N-dimethylacetamide (DMA) and the like; or mixtures thereof.

The amount of solvent used for dissolution may range from about 10-18 times, or about 10 times, the initial weight of the 2-[(2-amino-6-oxo-1,6-dihydor-9H-purin-9yl)methoxy]ethyl-N-[(benzyloxy)carbonyl]-L-valinate of Formula II.

Suitable temperatures for forming a solution range from about 25° C. to about 100° C., or the reflux temperature of the solvent used.

Step 2) involves cooling the solution of step 1) for a suitable time and to suitable temperatures, and/or adding a suitable antisolvent or mixture of antisolvents to the solution of step 1) to form crystals under suitable conditions.

For solid formation to occur, the mass may be maintained further at temperatures lower than the dissolution temperatures, such as for example below about 10° C. to about 25° C., for a period of time as required for a more complete isolation of the product. The exact cooling temperature and time required for complete crystallization may be readily determined by a person skilled in the art and will also depend on parameters such as concentration and temperature of the solution or slurry.

The time required for precipitation of solid may range from about 2 hours to about 10 hours, or longer, depending on the desired extent of product recovery.

Optionally the solvent can be partially evaporated to induce precipitation of desired solid, and then the mass may be cooled to obtain a higher yield.

Optionally, crystallization may be initiated by methods such as cooling, seeding, partial removal of the solvent from the solution, by adding an anti-solvent to the solution, or a combination thereof.

Suitable anti-solvents that can be used in step 2) include: water, alcoholic solvents such as methanol, ethanol, isopropyl alcohol, n-butanol, tertiary-butyl alcohol, and the like; ketonic solvents such as acetone, ethyl methyl ketone, methyl isobutyl ketone and the like; esters such as ethyl acetate, n-propyl acetate, isopropyl acetate, n-butyl acetate, tertiary-butyl acetate and the like; nitrile solvents such as acetonitrile, propionitrile and the like; halogenated solvents such as dichloromethane, ethylene dichloride, chloroform and the like; or mixtures thereof.

Step 3) involves recovering the solid of step 2) to afford the desired pure compound 2-[(2-amino-6-oxo-1,6-dihydor-9H-purin-9yl) methoxy]ethyl-N-[(benzyloxy)carbonyl]-L-valinate of Formula II.

The method by which the solid material is recovered from the final mixture, with or without cooling below the operating temperature, may be any of techniques such as decantation, filtration by gravity or by suction, centrifugation, and the like. The crystals so isolated typically carry a small proportion of occluded mother liquor containing a higher percentage of impurities. If desired the crystals may be washed with a solvent to wash out the mother liquor.

The solid obtained in step 3) may optionally be further dried.

Drying may be suitably carried out in equipment such as a tray dryer, vacuum oven, air oven, fluidized bed drier, spin flash dryer, flash dryer and the like. The drying may be carried out at temperatures of about 25° C. to about 75° C. The drying may be carried out for any desired time periods to achieve the desired product purity, times from about 1 to 20 hours, or longer, frequently being adequate.

In the event that a higher purity is required, the above crystallization process may be repeated one, two or more times, or the product may be slurried in a suitable organic solvent.

In another aspect, there is provided pure 2-[(2-amino-6-oxo-1,6-dihydro-9H-purin-9yl)methoxy]ethyl-N-[(benzyloxy)carbonyl]-L-valinate of Formula II having a purity greater than or equal to about 97% w/w as determined by chiral high performance liquid chromatography (“chiral HPLC”).

In yet another aspect, there are provided processes for the purification of valacyclovir hydrochloride of Formula I, an embodiment of a process including:

1 ) providing a solution of valacyclovir hydrochloride in a suitable solvent or mixture of solvents under suitable conditions;

2) cooling the reaction solution of step 1) to suitable temperatures for crystallization of the solid, and/or adding a suitable antisolvent or mixture of antisolvents to the solution of step 1) to form crystals under suitable conditions; and

3) recovering a solid formed in step 2) to afford the desired pure compound.

Step 1) involves providing a solution of valacyclovir hydrochloride in a suitable solvent or mixture of solvents under suitable conditions.

Suitable organic solvents include but are not limited to: water, alcoholic solvents such as methanol, ethanol, isopropyl alcohol, n-butanol, tertiary-butyl alcohol, and the like; ketonic solvents such as acetone, ethyl methyl ketone, methyl isobutyl ketone and the like; esters such as ethyl acetate, n-propyl acetate, isopropyl acetate, n-butyl acetate, tertiary-butyl acetate and the like; nitrile solvents such as acetonitrile, propionitrile and the like; halogenated solvents such as dichloromethane, ethylene dichloride, chloroform and the like; aprotic polar solvents such as N,N-dimethylformamide (DMF), dimethylsulfoxide (DMSO), N,N-dimethylacetamide (DMA) and the like; and mixtures thereof or their combinations with water in various proportions without limitation.

The amount of solvent used is not particularly limited. For enhanced product recovery, it will be used in an amount that forms a concentrated solution that is close to the solubility limit of valacyclovir hydrochloride in the solvent.

The amount of solvent used for dissolution may range from about 6-18 times or about 10 times the weight of the valacyclovir hydrochloride to be dissolved.

Step 2) involves cooling the solution of step 1), and/or saturating by adding a suitable antisolvent or mixture of antisolvents to form crystals under suitable conditions.

For solid formation to occur, the mass may be maintained further at temperatures lower than the dissolution temperatures, such as for example below about 10° C. to about 25° C., for a period of time as required for a more complete isolation of the product. The exact cooling temperature and time required for complete crystallization may be readily determined by a person skilled in the art and will also depend on parameters such as concentration and temperature of the solution or slurry.

The time required for precipitation of solid may range from about 2 hours to about 10 hours, or longer, depending on the desired extent of product recovery.

Optionally the solvent can be partially evaporated to induce precipitation of desired solid, and then the mass may be cooled to obtain a higher yield.

Optionally, crystallization may be initiated by methods such as cooling, seeding, partial removal of the solvent from the solution, by adding an anti-solvent to the solution, or a combination thereof.

Suitable anti-solvents that can be used in the step include: alcoholic solvents such as methanol, ethanol, isopropyl alcohol, n-butanol, tertiary-butyl alcohol, and the like; ketonic solvents such as acetone, ethyl methyl ketone, methyl isobutyl ketone and the like; esters such as ethyl acetate, n-propyl acetate, isopropyl acetate, n-butyl acetate, tertiary-butyl acetate and the like; nitrile solvents such as acetonitrile, propionitrile and the like; halogenated solvents such as dichloromethane, ethylene dichloride, chloroform and the like; or mixtures thereof.

Optionally the solvent may be partially evaporated to enhance precipitation of the desired solid.

The amount of anti-solvent used for solid precipitation may range from about 1-6 times, or about 3 times, the initial volume of the valacyclovir hydrochloride solution.

Step 3) involves recovering a solid formed in step 2) to afford the desired pure compound.

The method by which the solid material is recovered from the final mixture, with or without cooling below the operating temperature, may be any of techniques such as decantation, filtration by gravity or by suction, centrifugation, and the like. The crystals so isolated will carry a small proportion of occluded mother liquor containing a higher percentage of impurities. If desired the crystals may be washed with a solvent to wash out the mother liquor.

The solid obtained in step 3) may optionally be further dried.

Drying may be suitably carried out in equipment such as a tray dryer, vacuum oven, air oven, fluidized bed drier, spin flash dryer, flash dryer and the like. The drying may be carried out at temperatures of about 25° C. to about 75° C. The drying may be carried out for any desired time periods to achieve the desired product purity, times from about 1 to 20 hours, or longer, frequently being adequate.

In the event that a higher purity is required, the above recrystallization process may be repeated one, two or more times, or the product may be slurried in a suitable organic solvent having a low solubility for valacyclovir hydrochloride.

In an embodiment, there is provided pure valacyclovir hydrochloride of Formula I having a chiral purity greater than or equal to about 97% w/w, as determined using chiral HPLC.

In an embodiment, there is provided pure valacyclovir hydrochloride of Formula I, with a D-isomeric impurity content less than or equal to about 3% w/w, as determined using chiral HPLC.

In the description, certain conditions such as temperatures and certain operations such as filtration are described to illustrate the invention. Persons skilled in the art will be aware that other conditions and operations will also be suitable, and those are included within the invention. For example, operations such as centrifugation, decantation, etc. are acceptable substitutes for filtration.

The pharmaceutical compositions comprising valacyclovir or its pharmaceutically acceptable salt of the invention together with one or more pharmaceutically acceptable excipients may be formulated as: solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir systems or combinations of matrix and reservoir systems. The compositions may be prepared by direct blending, dry granulation or wet granulation or by extrusion and spheronization. Compositions may be presented as uncoated, film coated, sugar coated, powder coated, enteric coated or modified release coated. Compositions of the present invention may further comprise one or more pharmaceutically acceptable excipients.

Pharmaceutically acceptable excipients that are useful in the present invention include, but are not limited to: diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, polyvinylpyrrolidone, hydroxypropyl cellulose, hydroxypropyl methylcellulose, pregelatinized starch and the like; disintegrants such as starch, sodium starch glycolate, pregelatinized starch, crospovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins, resins; release rate controlling agents such as hydroxypropyl cellulose, hydroxymethyl cellulose, hydroxypropyl methylcellulose, ethyl cellulose, methylcellulose, various grades of methyl methacrylates, waxes and the like. Other pharmaceutically acceptable excipients that are of use include but are not limited to film formers, plasticizers, colorants, flavoring agents, sweeteners, viscosity enhancers, preservatives, antioxidants and the like.

The processes of the present invention are simple, cost effective, ecofriendly, reproducible, scalable, and robust to produce valacyclovir hydrochloride and its intermediates with high purity.

Certain specific aspects and embodiments of the present invention will be explained in greater detail with reference to the following examples, which are provided by way of illustration only and should not be construed as limiting the scope of the invention in any manner.

### EXAMPLE 1

### PURIFICATION OF 2-[(2-AMINO-6-OXO-1,6-DIHYDRO-9H-PURIN-9YL)METHOXY]ETHYL-N-[(BENZYLOXY)CARBONYL]-L-VALINATE USING ACETONE AND WATER

2-[(2-amino-6-oxo-1,6-dihydro-9H-purin-9yl)methoxy]ethyl-N-[(benzyloxy)carbonyl]-L-valinate (10 g) of Formula II (D-isomer content 3.5%) was charged into a clean and dry 4-neck round bottom flask containing acetone (120 ml) and water (30 ml). The reaction mixture was heated to a temperature of 60° C. followed by stirring for a period of 20-30 minutes to form a solution. The solution was cooled to a temperature of 25° C. The formed solid was filtered and the solid was washed with acetone (10 ml). The solid obtained was dried at a temperature of 60° C. under vacuum for a period of 5 hours to afford 8.1 g of the title compound. D-isomer content: 2.09% w/w by chiral HPLC.

### EXAMPLE 2

### PURIFICATION OF 2-[(2-AMINO-6-OXO-1,6-DIHYDRO-9H-PURIN-9YL)METHOXY]ETHYL-N-[(BENZYLOXY)CARBONYL]-L-VALINATE

2-[(2-amino-6-oxo-1,6-dihydro-9H-purin-9yl)methoxy]ethyl-N-[(benzyloxy)carbonyl]-L-valinate (20 g) of Formula II (D-isomer content 3%) was charged into a clean and dry round bottom flask containing N,N-dimethylformamide (100 ml). The mixture was stirred at a temperature of 30° C. for period of 15 minutes for complete dissolution. The resultant solution was cooled to a temperature of 15° C. pH of the solution was adjusted to about 3 by the addition of 36% hydrochloric acid (1.5 ml) followed by stirring for a period of 40 minutes. The obtained solution was divided into four parts, which were further treated as described below.

Part I

Above-obtained solution (28 ml) was charged into clean and dry round bottom flask at a temperature of 20-30° C. Water (140 ml) was added slowly through a dropper over a period of 20-30 minutes. The resultant mixture was stirred for a period of 20-30 minutes at a temperature of 25-30° C. The separated solid was filtered and washed with water (10 ml) and dried under a vacuum at a temperature of 60° C. to afford 4.3 g of the title compound with a D-isomeric impurity content of 2.73% w/w by chiral HPLC.

Part II

Above-obtained solution (28 ml) was charged into a fresh clean dry round bottom flask at a temperature of 20-30° C. The pH of the solution was adjusted to 1.2 by addition of 36% hydrochloric acid (4 ml) and stirred for a period of 10-12 hours. From the above-obtained solution 28 ml of solution was charged into a clean and dry round bottom flask and water (56 ml) was added slowly through a dropper over a period of 20-30 minutes. The resultant mixture was stirred for about 20-30 minutes at a temperature of 25-30° C. The formed solid was filtered and washed with water (10 ml) and dried under a vacuum to afford 3 g of the title compound with a D-isomeric impurity content of 2.73% w/w by chiral HPLC.

Part III

Above-obtained solution (28 ml) was charged into a fresh dry round bottom flask at a temperature of 20-30° C. A mixture of acetone (84 ml) and water (112 ml) was added slowly through a dropper over about 20-30 minutes. The resultant mixture was stirred for a period of 20-30 minutes at a temperature of 25-30° C. The separated solid was filtered and dried under vacuum at a temperature of 50-60° C. to afford 4 g of the title compound with a D-isomeric impurity content of 1.91% w/w by chiral HPLC.

Part IV

Above-obtained solution (28 ml) was charged into a fresh dry round bottom flask at a temperature of 20-30° C. A mixture of methanol (56 ml) and water (84 ml) was charged slowly through a dropper over a period of 20-30 minutes. The resultant mixture was stirred for a period of 20-30 minutes at a temperature of 25-30° C. The formed solid was filtered and washed with water (10 ml). The obtained solid was dried under vacuum at a temperature of 60-65° C. to afford 4.1 g of the title compound with a D-isomeric impurity content of 2.58% w/w by chiral HPLC.

### EXAMPLE 3

### PURIFICATION OF 2-[(2-AMINO-6-OXO-1,6-DIHYDRO-9H-PURIN-9YL)METHOXY]ETHYL-N-[(BENZYLOXY)CARBONYL]-L-VALINATE USING THF

2-[(2-amino-6-oxo-1,6-dihydro-9H-purin-9yl)methoxy]ethyl-N-[(benzyloxy)carbonyl]-L-valinate (3 g) of Formula II (D-isomer content 3%) was charged into a clean and dry round bottom flask containing THF (150 ml). The resultant mixture was heated to a temperature of 60-70° C. for complete dissolution and stirred for a period of 30-60 minutes. The solution was cooled to a temperature of 25-30° C. and stirred for a period of 10-15 minutes. The separated solid was filtered and washed with THF (3 ml) to afford 0.3 g of the title compound with a D-isomeric content of 1.48% w/w by chiral HPLC.

### EXAMPLE 4

### PURIFICATION OF 2-[(2-AMINO-6-OXO-1,6-DIHYDRO-9H-PURIN-9YL)METHOXY]ETHYL-N-[(BENZYLOXY)CARBONYL]-L-VALINATE USING METHANOL AND ACETONE

2-[(2-amino-6-oxo-1,6-dihydro-9H-purin-9yl)methoxy]ethyl-N-[(benzyloxy)carbonyl]-L-valinate (5 g) of Formula II (D-isomer content 3%) was charged into a clean and dry round bottom flask containing methanol (50 ml) and acetone (60 ml). The resultant mixture was heated to a temperature of 50-60° C. for complete dissolution and stirred for a period of 30-60 minutes. The obtained solution was distilled to about 50% of the initial volume. The solution was cooled to a temperature of 25-30° C. and stirred for solid formation. The solid was filtered and washed with acetone (5 ml) and suction dried for a period of 10 minutes. The obtained solid was dried under vacuum at a temperature of 60-65° C. to afford 2.9 g of the title compound with a D-isomeric content 1.85% w/w by chiral HPLC.

### EXAMPLE 5

### PURIFICATION OF 2-[(2-AMINO-6-OXO-1,6-DIHYDRO-9H-PURIN-9YL)METHOXY]ETHYL-N-[(BENZYLOXY)CARBONYL]-L-VALINATE USING WATER AND ACETONE

2-[(2-amino-6-oxo-1,6-dihydro-9H-purin-9yl)methoxy]ethyl-N-[(benzyloxy)carbonyl]-L-valinate (10 g) of Formula II (D-isomer content 3%) was charged into a clean and dry round bottom flask containing acetone (120 ml) and water (30 ml). The resultant mixture was heated to a temperature of 60-70° C. for complete dissolution and stirred for a period of 30-60 minutes. The resultant solution was cooled to a temperature of 15-20° C. followed by addition of water (30 ml) and stirred for a period of 10-15 minutes. The formed solid was filtered and washed with acetone (30 ml). The obtained solid was dried under vacuum at a temperature of 60-70° C. to afford 8.1 g of the title compound with a D-isomeric content 2.09% w/w by chiral HPLC.

### EXAMPLE 6

### PURIFICATION OF 2-[(2-AMINO-6-OXO-1,6-DIHYDRO-9H-PURIN-9YL)METHOXY]ETHYL-N-[(BENZYLOXY)CARBONYL]-L-VALINATE USING ACETONE AND WATER

2-[(2-amino-6-oxo-1,6-dihydro-9H-purin-9yl)methoxy]ethyl-N-[(benzyloxy)carbonyl]-L-valinate (5 g) of Formula II (D-isomer content 3%) was charged into a clean and dry round bottom flask containing acetone (150 ml) and water (15 ml). The resultant mixture was heated to a temperature of 60-70° C. for complete dissolution and stirred for a period of 30-60 minutes. The resultant solution was cooled to a temperature of 15-20° C. followed by addition of water (30 ml) and stirred for a period of 10-15 minutes. The formed solid was filtered and washed with acetone (30 ml). The obtained solid was dried under vacuum at a temperature of 60-70° C. to afford 3.2 g of the title compound with a D-isomeric content 1.92% w/w by chiral HPLC.

### EXAMPLE 7

### PURIFICATION OF 2-[(2-AMINO-6-OXO-1,6-DIHYDRO-9H-PURIN-9YL)METHOXY]ETHYL-N-[(BENZYLOXY)CARBONYL]-L-VALINATE USING A MIXTURE OF METHANOL, ETHANOL AND WATER

2-[(2-amino-6-oxo-1,6-dihydro-9H-purin-9yl)methoxy]ethyl-N-[(benzyloxy)carbonyl]-L-valinate (3 g) of Formula II (D-isomer content 3%) was charged into a clean and dry round bottom flask containing methanol (24 ml), ethanol (25 ml) and water (15 ml). The resultant mixture was heated to a temperature of 60-70° C. for complete dissolution and stirred for a period of 30-60 minutes. The resultant solution was cooled to a temperature of 15-20° C. followed by addition of water (30 ml) and stirred for a period of 10-15 minutes. The formed solid was filtered and washed with a mixture of acetone and ethanol (6 ml of each). The obtained solid was dried under vacuum at a temperature of 60-70° C. to afford 0.8 g of the title compound with a D-isomeric content 2.4% w/w by chiral HPLC.

### EXAMPLE 8

### PURIFICATION OF 2-[(2-AMINO-6-OXO-1,6-DIHYDRO-9H-PURIN-9YL)METHOXY]ETHYL-N-[(BENZYLOXY)CARBONYL]-L-VALINATE USING A MIXTURE OF METHANOL AND ACETONE

2-[(2-amino-6-oxo-1,6-dihydro-9H-purin-9yl)methoxy]ethyl-N-[(benzyloxy)carbonyl]-L-valinate (5 g) of Formula II (D-isomer content 3%) was charged into a clean and dry round bottom flask containing methanol (50 ml) and water (50 ml). The resultant mixture was heated to a temperature of 60-70° C. for complete dissolution and stirred for a period of 30-60 minutes. The resultant solution was cooled to a temperature of 25-30° C. and stirred for a period of 10-15 minutes. The formed solid was filtered and washed with acetone (5 ml). The obtained solid was suction dried to afford 2.5 g of the title compound with a D-isomeric content 1.92% w/w by chiral HPLC.

### EXAMPLE 9

### PURIFICATION OF 2-[(2-AMINO-6-OXO-1,6-DIHYDRO-9H-PURIN-9YL)METHOXY]ETHYL-N-[(BENZYLOXY)CARBONYL]-L-VALINATE USING A MIXTURE OF METHANOL, WATER AND ACETONE

2-[(2-amino-6-oxo-1,6-dihydro-9H-purin-9yl)methoxy]ethyl-N-[(benzyloxy)carbonyl]-L-valinate (5 g) of Formula II (D-isomer content 3%) was charged into a clean and dry round bottom flask containing methanol (50 ml) and water (25 ml). The resultant mixture was heated to a temperature of 60-70° C. for complete dissolution and stirred for a period of 30-60 minutes. The resultant solution was cooled to a temperature of 25-30° C. and stirred for a period of 10-15 minutes. The formed solid was filtered and washed with acetone (5 ml). The obtained solid was suction dried to afford 3.0 g of the title compound with a D-isomeric content 1.92% w/w by chiral HPLC.

### EXAMPLE 10

### PURIFICATION OF 2-[(2-AMINO-6-OXO-1,6-DIHYDRO-9H-PURIN-9YL)METHOXY]ETHYL-N-[(BENZYLOXY)CARBONYL]-L-VALINATE USING A MIXTURE OF METHANOL AND WATER

2-[(2-amino-6-oxo-1,6-dihydro-9H-purin-9yl)methoxy]ethyl-N-[(benzyloxy)carbonyl]-L-valinate (5 g) of Formula II (D-isomer content 3%) was charged into a clean and dry round bottom flask containing methanol (50 ml) and water (25 ml). The resultant mixture was heated to a temperature of 60-70° C. for complete dissolution and stirred for a period of 30-60 minutes. The resultant solution was cooled to a temperature of 25-30° C. and stirred for a period of 10-15 minutes. The formed solid was filtered and washed with acetone (5 ml). The obtained solid was suction dried to afford 3.0 g of the title compound with a D-isomeric content 2.61% w/w by chiral HPLC.

### EXAMPLE 11

### PURIFICATION OF 2-[(2-AMINO-6-OXO-1,6-DIHYDRO-9H-PURIN-9YL)METHOXY]ETHYL-N-[(BENZYLOXY)CARBONYL]-L-VALINATE USING METHANOL

2-[(2-amino-6-oxo-1,6-dihydro-9H-purin-9yl)methoxy]ethyl-N-[(benzyloxy)carbonyl]-L-valinate (5 g) of Formula II (D-isomer content 3%) was charged into a clean and dry round bottom flask containing methanol (150 ml). The resultant mixture was heated to a temperature of 60-70° C. for complete dissolution and stirred for a period of 30-60 minutes. The resultant solution was cooled to a temperature of 25-30° C. and stirred for a period of 10-15 minutes. The formed solid was filtered and washed with methanol (5 ml). The obtained solid was dried under vacuum at a temperature of 60-70° C. to afford 4.3 g of the title compound with a D-isomeric content 2.2% w/w by chiral HPLC.

### EXAMPLE 12

### PURIFICATION OF 2-[(2-AMINO-6-OXO-1,6-DIHYDRO-9H-PURIN-9YL)METHOXY]ETHYL-N-[(BENZYLOXY)CARBONYL]-L-VALINATE USING A MIXTURE OF METHANOL AND WATER

2-[(2-amino-6-oxo-1,6-dihydro-9H-purin-9yl)methoxy]ethyl-N-[(benzyloxy)carbonyl]-L-valinate (5 g) of Formula II (D-isomer content 3%) was charged into a clean and dry round bottom flask containing methanol (75 ml) and water (1.2 ml). The resultant mixture was heated to a temperature of 60-70° C. for complete dissolution and stirred for a period of 30-60 minutes. The resultant solution was cooled to a temperature of 25-30° C. and stirred for a period of 10-15 minutes. The formed solid was filtered and washed with methanol (5 ml). The obtained solid was dried under vacuum at a temperature of 60-65° C. to afford 4.3 g of the title compound with a D-isomeric content 2.3% w/w by chiral HPLC.

### EXAMPLE 13

### PURIFICATION OF 2-[(2-AMINO-6-OXO-1,6-DIHYDRO-9H-PURIN-9YL)METHOXY]ETHYL-N-[(BENZYLOXY)CARBONYL]-L-VALINATE USING METHANOL

2-[(2-amino-6-oxo-1,6-dihydro-9H-purin-9yl)methoxy]ethyl-N-[(benzyloxy)carbonyl]-L-valinate (5 g) of Formula II (D-isomer content 3%) was charged into a clean and dry round bottom flask containing methanol (150 ml). The resultant mixture was heated to a temperature of 60-70° C. for complete dissolution and stirred for a period of 30-60 minutes. The obtained solution was distilled to 80% of its original volume. The obtained solution was cooled to a temperature of 25-30° C. and stirred for solid formation. The solid was filtered and washed with methanol (5 ml) and suction dried for a period of 10 minutes. The obtained solid was dried under vacuum at a temperature of 60-70° C. to afford 3.8 g of the title compound with a D-isomeric content 2.3% w/w by chiral HPLC.

### EXAMPLE 14

### PURIFICATION OF VALACYCLOVIR HYDROCHLORIDE USING ACETONITRILE AND WATER

Valacyclovir hydrochloride (50 g) (3.5% D-isomeric impurity content) were charged into a clean and dry round bottom flask containing acetonitrile (375 ml) and water (107 ml). The contents were heated to a temperature of 70° C. and stirred for a period of 30 minutes followed by cooling the formed solution to a temperature of 30° C. To the resultant solution, acetonitrile (143 ml) was added slowly at a temperature of 30° C. over a period of 30 minutes followed by stirring for a period of 30 minutes for solid formation. The solid was filtered and was washed with acetonitrile (50 ml). The solid obtained was dried at a temperature of 60° C. under vacuum for a period of 5 hours to afford 38.7 g of the title compound.

D-isomer content: 2.84% w/w by chiral HPLC.

Purity by chiral HPLC: 97.16%.

### EXAMPLE 15

### PURIFICATION OF VALACYCLOVIR HYDROCHLORIDE USING ACETONITRILE

Valacyclovir hydrochloride (3.5% w/w D-isomer content) (50 g) was charged into a clean and dry round bottom flask containing acetonitrile (500 ml). The contents were heated at a temperature of 65° C. followed by stirring for a period of 30 minutes. The resultant solution was cooled to a temperature of 30° C. To the solution, acetonitrile (200 ml) was added slowly over a period of 30 minutes followed by stirring for a period of 30 minutes. The formed solid was filtered and was washed with acetonitrile (50 ml). The solid obtained was dried at a temperature of 60° C. under vacuum for a period of 5 hours to afford 38.7 g of the title compound.

D-isomer content: 2.67% w/w by chiral HPLC.

Purity by chiral HPLC: 97.33%.

### EXAMPLE 16

### PURIFICATION OF VALACYCLOVIR HYDROCHLORIDE USING ETHANOL

Valacyclovir hydrochloride (2 g) (3.5% w/w of D-isomer) was charged into a clean and dry round bottom flask containing ethanol (20 ml). The contents were heated to a temperature of 65° C. followed by stirring for a period of 30 minutes. To the resultant solution, water (6 ml) was added slowly over a period of 10 minutes followed by stirring for a period of 30 minutes. The solution was cooled to a temperature of 30° C. and stirred for a period of 20-30 minutes. The formed solid was filtered and was washed with ethanol (5 ml). The solid obtained was dried at a temperature of 60° C. under vacuum for a period of 1 hour to afford 1.2 g of the title compound. D-isomer content: 2.67% w/w by chiral HPLC.

We claim:

1. A process for purifying 2-[(2-amino-6-oxo-1,6-dihydro-9H-purin-9yl)methoxy]ethyl-N-[(benzyloxy)carbonyl]-L-valinate having the formula: 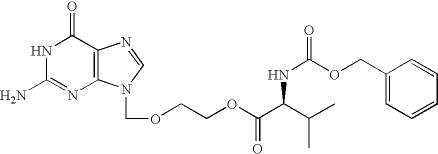
comprising:
a) providing a solution of 2-[(2-amino-6-oxo-1,6-dihydro-9H-purin-9-yl)methoxy]ethyl-N-[(benzyloxy)carbonyl]-L-valinate in a solvent;
b) cooling the solution of step a) for crystallization of a solid, or adding an antisolvent to the solution of step a) to form crystals, or both; and
c) recovering a solid formed in step b).

2. The process of claim 1, wherein a solvent comprises methanol, ethanol, isopropyl alcohol, n-butanol, tertiary-butyl alcohol, acetone, ethyl methyl ketone, methyl isobutyl ketone, ethyl acetate, n-propyl acetate, isopropyl acetate, n-butyl acetate, tertiary-butyl acetate, acetonitrile, propionitrile, dichloromethane, ethylene dichloride, chloroform, N,N-dimethylformamide, dimethylsulfoxide, N,N-dimethylacetamide, or mixtures thereof.

3. The process of claim 1, wherein an anti-solvent comprises water, methanol, ethanol, isopropyl alcohol, n-butanol, tertiary-butyl alcohol, acetone, ethyl methyl ketone, methyl isobutyl ketone, ethyl acetate, n-propyl acetate, isopropyl acetate, n-butyl acetate, tertiary-butyl acetate, acetonitrile, propionitrile, dichloromethane, ethylene dichloride, chloroform, or mixtures thereof.

4. The process of claim 1, wherein the obtained solid is 2-[(2-amino-6-oxo-1,6-dihydro-9H-purin-9-yl)methoxy]ethyl-N-[(benzyloxy)carbonyl]-L-valinate having a chiral purity at least about 97% w/w.

5. A process for purifying valacyclovir hydrochloride, comprising:
a) providing a solution of valacyclovir hydrochloride in a solvent;
b) cooling the solution of step a) for crystallization of a solid, or adding an antisolvent to the solution of step a) to form crystals, or both; and
c) recovering a solid formed in step b).

6. The process of claim 5, wherein a solvent comprises water, methanol, ethanol, isopropyl alcohol, n-butanol, tertiary-butyl alcohol, acetone, ethyl methyl ketone, methyl isobutyl ketone, ethyl acetate, n-propyl acetate, isopropyl acetate, n-butyl acetate, tertiary-butyl acetate, acetonitrile, propionitrile, dichloromethane, ethylene dichloride, chloroform, N,N-dimethylformamide, dimethylsulfoxide, N,N-dimethylacetamide, mixtures thereof, or their combinations with water.

7. The process of claim 5, wherein an anti-solvent comprises methanol, ethanol, isopropyl alcohol, n-butanol, tertiary-butyl alcohol, acetone, ethyl methyl ketone, methyl isobutyl ketone, ethyl acetate, n-propyl acetate, isopropyl acetate, n-butyl acetate, tertiary-butyl acetate, acetonitrile, propionitrile, dichloromethane, ethylene dichloride, chloroform, mixtures thereof.

8. A process for the purification of valacyclovir hydrochloride comprising:
a) providing a solution of 2-[(2-amino-6-oxo-1,6-dihydro-9H-purin-9-yl)methoxy]ethyl-N-[(benzyloxy)carbonyl]-L-valinate in a solvent;
b) cooling the reaction solution of step a) for crystallization of the solid, or adding a antisolvent or mixture of antisolvents to solution of step a) to form crystals, or both;
c) recovering the solid formed in step b);
d) converting the solid of step c) into valacyclovir hydrochloride;
e) providing a solution of valacyclovir hydrochloride in a suitable solvent;
f) cooling the reaction solution of step e) for crystallization of the solid, or adding a antisolvent or mixture of antisolvents to solution of step e) to form crystals, or both; and
g) recovering a solid formed in step f).

9. The process of claim 8, wherein a solvent in step a) comprises methanol, ethanol, isopropyl alcohol, n-butanol, tertiary-butyl alcohol, acetone, ethyl methyl ketone, methyl isobutyl ketone, ethyl acetate, n-propyl acetate, isopropyl acetate, n-butyl acetate, tertiary-butyl acetate, acetonitrile, propionitrile, dichloromethane, ethylene dichloride, chloroform, N,N-dimethylformamide, dimethylsulfoxide, N,N-dimethylacetamide, or mixtures thereof.

10. The process of claim 8, wherein an anti-solvent in step b) comprises water, methanol, ethanol, isopropyl alcohol, n-butanol, tertiary-butyl alcohol, acetone, ethyl methyl ketone, methyl isobutyl ketone, ethyl acetate, n-propyl acetate, isopropyl acetate, n-butyl acetate, tertiary-butyl acetate, acetonitrile, propionitrile, dichloromethane, ethylene dichloride, chloroform or mixtures thereof.

11. The process of claim 8, wherein a solvent in step e) comprises water, methanol, ethanol, isopropyl alcohol, n-butanol, tertiary-butyl alcohol, acetone, ethyl methyl ketone, methyl isobutyl ketone, ethyl acetate, n-propyl acetate, isopropyl acetate, n-butyl acetate, tertiary-butyl acetate, acetonitrile, propionitrile, dichloromethane, ethylene dichloride, chloroform, N,N-dimethylformamide, dimethylsulfoxide, N,N-dimethylacetamide, or mixtures thereof.

12. The process of claim 8, wherein an anti-solvent in step f) comprises methanol, ethanol, isopropyl alcohol, n-butanol, tertiary-butyl alcohol, acetone, ethyl methyl ketone, methyl isobutyl ketone, ethyl acetate, n-propyl acetate, isopropyl acetate, n-butyl acetate, tertiary-butyl acetate, acetonitrile, propionitrile, dichloromethane, ethylene dichloride, chloroform or mixtures thereof.

13. Valacyclovir hydrochloride, prepared by the process of claim 5.

14. Valacyclovir hydrochloride, prepared by the process of claim 8.
